(19) United States
(12) Patent Application Publication
(10) Pub. No.: US 2008/0221063 A1
(43) Pub. Date: Sep. 11, 2008

(54) Heterocyclic Sulfonamide Derivatives As Inhibitors Of Factor Xa

(75) Inventors: Christer Alstermark, Molndal (SE); Kjell Andersson, Molndal (SE); Ulf Fahlander, Molndal (SE); Kenneth Granberg, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/994,847

(22) PCT Filed: Jul. 5, 2006
(86) PCT No.: PCT/SE06/00836

§ 371 Date: Jan. 7, 2008

(30) Foreign Application Priority Data
Jul. 8, 2005 (SE) 0501615-9

(51) Int. Cl. A61K 31/715; C07D 403/14; A61K 31/501

(52) U.S. Cl. 514/54; 544/238; 514/252.02

(57) ABSTRACT

The invention relates to heterocyclic derivatives of formula (I), Chemical formula should be inserted here. Please see paper copy wherein R¹ is hydrogen or C₁₋₃alkyl; R² is selected from hydroxy, C₁₋₅alkyl, carboxy, cyano, tetrazolyl, N—C₁₋₅alkyltetrazolyl, oxazolyl, C₁₋₅oxazolyl, isoxazolyl, C₁₋₅ isoxazolyl, hydroxyC₁₋₅alkyl, carboxy C₁₋₅alkyl, C₁₋₅alkoxyoxo C₁₋₅alkyl, carbamoyl, C₁₋₅alkylcarbamoyl, di(C₁₋₅alkyl)carbamoyl, C₁₋₅alkylcarbamoyl C₁₋₄alkyl, hydroxy C₁₋₅alkylcarbamoyl, C₁₋₅alkoxy C₁₋₅alkylcarbamoyl; —C₁₋₅alkyl-Y¹, —COOCHR¹⁷R¹⁸ and —CONR¹⁷R¹⁸; and R³ is hydrogen or halogen; or a pharmaceutically acceptable salt thereof, said compounds possess antithrombotic and anticoagulant properties and are accordingly useful in methods of treatment of humans or animals. The invention also relates to processes for the preparation of the compounds, to their use, to pharmaceutical compositions comprising them, to their use in the manufacture of medicaments for use in the production of an antithrombotic or anticoagulant effect, and to combinations comprising them.

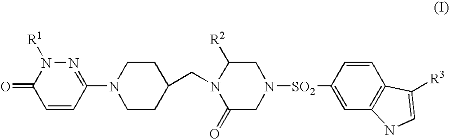

The invention relates to novel heterocyclic derivatives, or pharmaceutically-acceptable salts thereof, which possess antithrombotic and anticoagulant properties and are accordingly useful in methods of treatment of humans or animals. The invention also relates to processes for the preparation of the heterocyclic derivatives, to their use, to pharmaceutical compositions comprising them, to their use in the manufacture of medicaments for use in the production of an antithrombotic or anticoagulant effect, and to combinations comprising them.

The antithrombotic and anticoagulant effect produced by the compounds of the invention is believed to be attributable to their strong inhibitory effect against the activated coagulation protease known as Factor Xa. Factor Xa is one of a cascade of proteases involved in the complex process of blood coagulation. The protease known, as thrombin is the final protease in the cascade and Factor Xa is the preceding protease which cleaves prothrombin to generate thrombin.

Certain compounds are known to possess Factor Xa inhibitory properties and the field has been reviewed by B.-Y. Zhu, R. M. Scarborough, Current Opinion in Cardiovascular, Pulmonary & Renal Investigational Drugs, 1999, 1(1), 63-88. Thus it is known that two proteins, one known as recombinant antistasin (r-ATS) and the other known as recombinant tick anticoagulant protein (r-TAP), are specific direct Factor Xa inhibitors which possess antithrombotic properties in various animal models of thrombotic disease.

It is also known that certain non-peptidic compounds possess Factor Xa inhibitory properties. Of the low molecular weight inhibitors mentioned in the review by B.-Y. Zhu and R. M. Scarborough, many inhibitors possess a strongly basic group such as an amidinophenyl or amidinonaphthyl group.

We have now found that certain heterocyclic derivatives possess Factor Xa inhibitory activity. Many of the compounds of the present invention also possess the advantage of being selective Factor Xa inhibitors, that is the enzyme Factor Xa is inhibited strongly at concentrations of test compound which do not inhibit or which inhibit to a lesser extent the enzyme thrombin which is also a member of the blood coagulation enzymatic cascade.

The compounds of the present invention possess activity useful in the treatment or prevention of a variety of medical disorders where anticoagulant therapy is indicated, for example in the treatment or prevention of thrombotic conditions such as coronary artery and cerebrovascular disease. Further examples of such medical disorders include various cardiovascular and cerebrovascular conditions such as myocardial infarction, the rupture of atherosclerotic plaques, venous or arterial thrombosis, coagulation syndromes, vascular injury including reocclusion and restenosis following angioplasty and coronary artery bypass surgery, thrombus formation after the application of blood vessel operative techniques or after general surgery such as hip replacement surgery, the introduction of artificial heart valves or on the recirculation of blood, cerebral infarction, cerebral thrombosis, stroke, cerebral embolism, pulmonary embolism, ischemia and angina (including unstable angina).

The compounds of the invention are also useful as inhibitors of blood coagulation in an ex vivo situation such as, for example, the storage of whole blood or other biological samples suspected to contain Factor Xa and in which coagulation is detrimental.

WO 98/21188 describes a range of Factor Xa inhibitors. Further particular examples of this type of compound including 1-(5-chloroindol-2-ylsulphonyl)-4-[4-(6-oxo-1H-pyridazin-3-yl)benzoyl]piperazine are described in WO 99/57113. The applicants have found however, that by further derivatizing the compounds of this type, enhanced properties may be obtained.

The present invention provides a compound of formula (I)

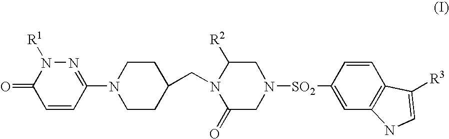

wherein R¹ is hydrogen or C₁₋₃alkyl;
R² is selected from hydroxy, C₁₋₅alkyl, carboxy, cyano, tetrazolyl, N—C₁₋₅ alkyltetrazolyl, oxazolyl, C₁₋₅ oxazolyl, isoxazolyl, C₁₋₅ isoxazolyl, hydroxyC₁₋₅alkyl, carboxyC₁₋₅alkyl, C₁₋₅alkoxyoxoC₁₋₅alkyl, carbamoyl, C₁₋₅alkylcarbamoyl, di(C₁₋₅alkyl)carbamoyl, C₁₋₅alkylcarbamoylC₁₋₄alkyl, hydroxyC₁₋₅alkylcarbamoyl, C₁₋₅alkoxyC₁₋₅alkylcarbamoyl; —C₁₋₅alkyl-Y¹, —COOCHR¹⁷R¹⁸ and —CONR¹⁷R¹⁸:

wherein

Y¹ represents O(CH₂)ᵣR¹⁴,

r represents an integer 1 to 4;

when r represents an integer 2 to 4, R¹⁴ represents hydroxy, C₁₋₅alkylalkoxy, carboxy, C₁₋₅alkoxycarbonyl, S(O)ₚR⁹ or NR¹⁵R¹⁶; and when r represents 1, R¹⁴ represents carboxy or C₁₋₅alkoxycarbonyl;

wherein any phenyl group within R² is independently substituted by 0, 1 or 2 substituents selected from halogeno, trifluoromethyl, cyano, C₁₋₅alkyl and C₁₋₅alkoxy;

p is 0, 1 or 2;

R⁹ represents C₁₋₅alkyl or phenyl;

R¹⁵ and R¹⁶ independently represent hydrogen or C₁₋₅alkyl;

R¹⁷ and 111 are independently selected from hydrogen, C₁₋₆allyl, C₄₋₇cycloalkyl, C₂₋₆alkenyl, R¹⁷ and R¹⁸ may form, along with the carbon to which they are attached, a 4-, 5-, 6- or 7-membered carbocyclic ring which contains 0, 1 or 2 heteroatoms selected from nitrogen, oxygen and sulphur, or R¹⁷ and R¹⁸ may form, along with the nitrogen to which they are attached, a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl; and

R³ is hydrogen or halogen;

or a pharmaceutically acceptable salt thereof.

In this specification the term “alkyl” includes both straight and branched chain alkyl groups but references to individual allyl groups such as “propyl” are specific for the straight chain version only. An analogous convention applies to other generic terms. For the avoidance of doubt, the atoms of the indolyl ring appearing in formula (I) is numbered as drawn below:

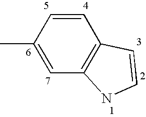

6-indolyl

It is to be understood that certain of the compounds of the formula (I) defined above can exist in solvated as well as unsolvated forms such as, for example, hydrated forms. It is to be understood that the invention encompasses all such solvated forms, which possess Factor Xa inhibitory activity.

It is further to be understood that, insofar as certain of the compounds of the formula (I) defined above may exist in optically active or racemic forms by virtue of one or more asymmetric carbon atoms, the invention encompasses any such optically active or racemic form which possesses Factor Xa inhibitory activity. The synthesis of optically active forms may be carried out by standard techniques of organic chemistry well known in the art, for example by synthesis from optically active starting materials or by resolution of a racemic form.

Further, “tautomer” or “tautomerism” refers to the coexistence of two (or more) compounds that differ from each other only in the position of one (or more) mobile atoms and in electron distribution, i.e. different tautomeric forms. An example may be keto-enol tautomers.
Moreover, it is also to be understood that, insofar as certain of the compounds of the formula (I) defined above may exist in various tautomeric forms, the invention encompasses any such tautomeric forms which possesses Factor Xa inhibitory activity.

Compounds of the invention are potent inhibitors of Factor Xa, and may have improved selectivity over oxido squalene cyclase, better solubility and/or less cytochrome P 450 (CYP₄₅₀) inhibition and/or Caco2-permeability than some related compounds. Caco2 is a cell line which mimics transport over the gut wall.

Suitable values in the compound of formula (I):
for halogen: fluoro, chloro, bromo, iodo;
for C₁₋₃alkyl (also as in e.g. oxoC₁₋₃alkyl): methyl, ethyl, propyl, isopropyl;
for C₁₋₄alkyl (also as in e.g. oxoC₁₋₄alkyl): methyl, ethyl, propyl, isopropyl, n-butyl, secbutyl, isobutyl, tertbutyl;
for C₁₋₅alkyl (also as in e.g. oxoC₁₋₅alkyl): C₁₋₄alkyl (as above), C₁₋₃alkyl (as above), n-butyl, isobutyl, pentyl, 2-pentyl, 3-pentyl, 2-methyl-1-butyl, isopentyl, neopentyl, 3-methyl-2-butyl, 2-methyl-2-butyl;
for C₁₋₃alkoxy: methoxy, ethoxy, propoxy, isopropoxy;
for C₁₋₄alkoxy: C₁₋₃alkoxy (as above), n-butoxy, secbutyl, isobutoxy, tertbutyl;
for C₁₋₅alkoxy: C₁₋₄alkoxy (as above), C₁₋₃alkoxy (as above), pentoxy, 2-pentoxy, 3-pentoxy, 2-methyl-1-butoxy, isopentoxy, neopentoxy, 3-methyl-2-butoxy, 2-methyl-2-butoxy;
for 4-, 5-, 6- or 7-membered heterocyclic ring: azetidine, pyrrolidine, morpholine, piperazine, azepane, [1,4]-diazepane, tetrahydro-pyran, or piperidin.
   Moreover, the term “oxido” denotes a ⁻O-group (ion) and the term “carbamoyl” denotes a H₂N—C(O)-group.
   In an embodiment of the invention a compound of formula (I) is disclosed where R¹ is C₁₋₃alkyl e.g. methyl, ethyl, or propyl.
   A further embodiment of the invention discloses a compound of formula (I) wherein R¹ is methyl.
   A further embodiment of the invention discloses a compound of formula (I) wherein R² is selected from hydroxy, C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —C₁₋₅alkyl-Y¹, —COOCHR¹⁷R¹⁸ and —CONR¹⁷R¹⁸:
   wherein Y¹ represents O(CH₂)ᵣR¹⁴;
   r represents an integer 1 to 4;
   when r represents an integer 2 to 4, R¹⁴ represents hydroxy, C₁₋₅alkylalkoxy, carboxy, C₁₋₅alkoxycarbonyl, S(O)ₚR⁹ or NR¹⁵R¹⁶; and when r represents 1, R¹⁴ represents carboxy or C₁₋₅alkoxycarbonyl;
   wherein any phenyl group within R¹ is independently substituted by 0, 1 or 2 substituents selected from halogeno, trifluoromethyl, cyano, C₁₋₅alkyl and C₁₋₅alkoxy;
   p is 0, 1 or 2;
   R⁹ represents C₁₋₅alkyl or phenyl;
   R¹⁵ and R¹⁶ independently represent hydrogen or C₁₋₅alkyl;

R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁵ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl carboxyC₁₋₅alkyl, C₁₋₅alkoxyoxoC₁₋₆allyl, and carbamoylC₁₋₅alkyl.

In a further embodiment of the invention a compound of formula (I) is disclosed where R² is selected from hydroxy, C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —COOCHR¹⁷R¹⁵ and —CONR¹⁷R¹⁵:
wherein
   R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and 118 may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹¹ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.
   An even further embodiment of the invention discloses a compound of formula (I) wherein 12 is selected from carboxy, hydroxyC₁₋₅alkyl, C₁₋₅alkoxyoxoC₁alkyl, carbamoyl, C₁₋₅alkylcarbamoyl, di(C₁₋₅allyl)carbamoyl, hydroxyC₁₋₅alkylcarbamoyl and C₁₋₅alkoxyC₁₋₅alkylcarbamoyl.
   A still further embodiment of the invention discloses a compound of formula (I) wherein R² is selected from
   —COOCHR¹⁷R¹⁸ and —CONR¹⁷R¹⁸: R¹⁷ and R¹⁸ are independently selected from hydrogen, C₁₋₆alkyl C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0 or 1 additional hetero oxygen, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl and carbamoylC₁₋₅alkyl.
   A further embodiment of the invention discloses a compound of formula (I) wherein R³ is halogen, e.g. fluoro, chloro or bromo.
   Said heterocyclic ring formed from R¹⁷ and R¹⁸ is, for example, azetidine, pyrrolidine, morpholine, piperazine, azepane, [1,4]-diazepane, tetrahydro-pyran, or piperidin.
   A further embodiment of the invention discloses a compound of formula (I) which is:
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid dimethylamide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethylamide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-hydroxy-ethyl)-amide,
6-{4-[4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
6-{4-[(R)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
6-{4-[(S)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide,
(S)-4-(3-Chloro-1H-indole-6-sulfonyl)-1-[1-(1-methyl-6-oxo-1,6-dihydro pyridazin-3-yl)-piperidin-4-ylmethyl]-6-oxo-piperazine-2-carboxylic acid isopropylamide,
6-{4-[2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[(R)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[(S)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[4-(3-Chloro-1H-indole-6-sulfonyl)-2-hydroxymethyl-6-oxo-piperazin-1-ylmethyl]-piperidine-1-yl}-2-methyl-2H-pyridazin-3-one,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
(S)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid tert-butyl ester,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethyl ester, or
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropyl ester.

A heterocyclic derivative of formula I, or pharmaceutically acceptable salt thereof, may be prepared by any process known to be applicable to the preparation of related compounds, such as those described in WO 98/21188 and WO 99/57113. Such procedures are provided as a further feature of the invention and are illustrated by the following representative processes in which, unless otherwise stated any functional group, for example amino, aminoalkyl, carboxy, indolyl or hydroxy, is optionally protected by a protecting group which may be removed when necessary.

Necessary starting materials may be obtained by standard procedures of organic chemistry and by reference to the processes used in the Examples.

The invention also relates to a process for preparing a compound of formula (I) which in an embodiment, amide derivatives from the exocyclic carboxylic acid of formula (I), or a reactive derivative thereof,

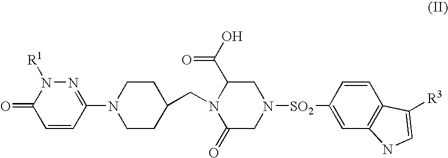

wherein the R-groups are as defined above in relation to formula (I), are prepared using an primary or secondary amine or a salt thereof.

A suitable reactive derivative of an acid of the formula (II) is, for example, an acyl halide, for example an acyl chloride formed by the reaction of the acid and an inorganic acid chloride, for example thionyl chloride; a mixed anhydride, for example an anhydride formed by the reaction of the acid with a chloroformate such as isobutyl chloroformate or with an activated amide such as 1,1′-carbonyldiimidazole; an active ester, for example an ester formed by the reaction of the acid and a phenol such as pentafluorophenol, an ester such as pentafluorophenyl trifluoroacetate or an alcohol such as N-hydroxybenzotriazole or N-hydroxysuccinimide; an acyl azide, for example an azide formed by the reaction of the acid and an azide such as diphenylphosphoryl azide; an acyl cyanide, for example a cyanide formed by the reaction of an acid and a cyanide such as diethylphosphoryl cyanide; or the product of the reaction of the acid and a carbodiimide such as N,N′dicyclohexylcarbodiimide or N-(3 dimethylamino-propyl)N′ethyl-carbodiimide.

The reaction is conveniently carried out in the presence of a suitable base such as, for example, an alkali or alkaline earth metal carbonate, also preferably carried out in a suitable inert solvent or diluent, for example methylene chloride or N,N-dimethylformamide, and at a temperature in the range, for example, −78° C. to 150° C., conveniently at or near ambient temperature.

In an alternative embodiment, ester derivatives from the exocyclic carboxylic acid of formula (II) or a reactive derivative thereof, wherein the R— groups are as defined above in relation to formula (I), are prepared using standard conditions following references found in Comprehensive Organic Transformations by Richard C. Larock. For example, for example treatment of (II) in an readily available alcoholic solvent using acid catalysis, for example, using by saturation of the solvent by gaseous hydrogen chloride, furnish the corresponding ester derivatives. In case of hindered alcohols N,N-dimethylformamide dialkyl acetal is useful.

The preparation of derivatives of formula (I) are prepared by reaction a sulfonyl chloride derivative of formula (III), with or without a protecting group on the indolyl nitrogen,

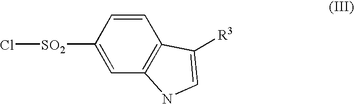

with an amine of formula (IV) or a salt thereof,

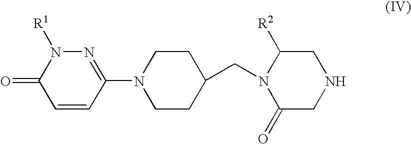

wherein the R-groups are as defined above in relation to formula (I).

This reaction is carried out using a base such as N,N-dimethyl aminopyridine, diisopropylethyl amine in inert solvents, typically dichloromethane and N,N-dimethylformamide at a temperature in the range −50° C.-100° C., conveniently at or near ambient temperature.

The preparation of derivatives of formula (V), wherein the R— groups are as defined above in relation to formula (I),

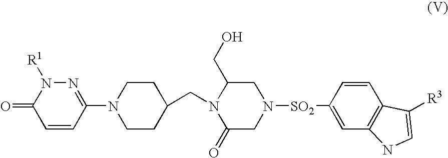

are prepared by reaction of a carboxylic acid derivative of formula (IV), or a reactive intermediate thereof e.g. a mixed anhydride formed by reacting (IV) with an alkyl chloroformate in situ, followed by addition of a reducing agent e.g. sodium borohydride.

This reaction is carried out in inert solvents, typically tetrahydrofuran at a temperature in the range −75° C.-50° C.

When a pharmaceutically-acceptable salt of a compound of the formula (I) is required, it may be obtained, for example, by reaction of said compound with a suitable acid or base using a conventional procedure.

When an optically active form of a compound of the formula (I) is required, it may be obtained, for example, by carrying out one of the aforesaid procedures using an optically active starting material or by resolution of a racemic form of said compound using a conventional procedure, for example by the formation of diastereomeric salts, use of chromatographic techniques, conversion using stereospecific enzymatic processes, or by addition of temporary extra chiral group to aid separation.

The invention also relates to a process for preparing a compound of formula (I) which process comprises either
(a) where an amide derivative from the exocyclic carboxylic acid of formula (II),

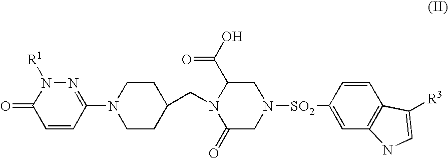

or a reactive derivative thereof, is prepared, using an primary or secondary amine or a salt thereof, and reacting with and an inorganic acid chloride
(b) where the compound of formula (I) is an ester derivative of the compound of formula (II), the compound of formula (II) is treated in an readily available alholic solvent using acid catalysis, for example, by using saturation of the solvent by gaseous hydrogen chloride, and using in the case of hindered alcohols N,N-dimethylformamide dialkyl acetal;
(c) reacting a sulfonyl chloride derivative of formula (III), with our without a protecting group on the indolyl nitrogen,

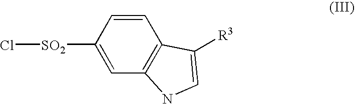

with an amine of formula (IV),

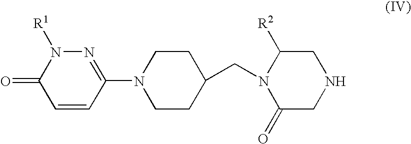

or a salt thereof; or
(d) reacting a carboxylic acid derivative of formula (IV), or a reactive intermediate thereof e.g. a mixed anhydride formed by reacting (IV) with an alkyl chloroformate in situ, followed by addition of a reducing agent.

As stated previously, the compounds of the formula (I) are inhibitors of the enzyme Factor Xa. The effects of this inhibition may be demonstrated using one or more of the standard procedures set out hereinafter:—

### a) Measurement of Factor Xa Inhibition

The FXa inhibitor potency was measured with a chromogenic substrate method, in a Plato 3300 robotic microplate processor (Rosys AG, CH-8634 Hombrechtikon, Switzerland), using 96-well, half-volume microtiter plates (Costar, Cambridge, Mass., USA; Cat No 3690). Stock solutions of test substance in DMSO (72 μL), 10 mmol/L, alternatively 1 mmol/L were diluted serially 1:3 (24+48 μL) with DMSO to obtain ten different concentrations, which were analyzed as samples in the assay, together with controls and blanks. As control sample melagatran was analysed. The dilutions of each test substance were analyzed consecutively, row-wise on the microtiter plate, with wasp cycles between substances to avoid cross-contamination. First 2 μL of test sample or DMSO for the blank were added, followed by 124 μL of assay buffer (0.05 mol/L Tris-hydrochloric acid pH 7.4 at 37° C., 5 mM CaCl₂, ionic strength 0.15 adjusted with NaCl, 0.1% bovine serum albumin, ICN Biomedicals, Inc, USA, 1 g/L) and 12 μL of chromogenic substrate solution (S-2765, Chromogenix, Mölndal, Sweden) and finally 12 μL of FXa solution (human FXa, Haematologic Technologies Inc., Essec Junction, Vt., USA), in buffer, was added, and the samples were mixed. The final assay concentrations were: test substance 0.0068-133, respectively 0.00068-13.3 μmol/L, S-2765 0.40 mmol/L (K_{M}=0.25 mmol/L) and FXa 0.1 mmol/L. The linear absorbance increase at 405 n=during 40 min incubation at 37° C. was used for calculation of percent inhibition for the test samples, as compared to references without inhibitor and/or enzyme. The IC₅₀-value, corresponding to the inhibitor concentration, which caused 50% inhibition of the FXa activity, was calculated by fitting the data to a three-parameter equation by Microsoft XLfit.

### b) Measurement of Thrombin Inhibition

The thrombin inhibitor potency was measured with a chromogenic substrate method developed in-house in principle as described in a) for FXa but using instead 0.3 nm of the chromogenic substrate solution S-2366 (Cliromogenix, Mölndal, Sweden) and 0.1 mmol/L human thrombin (Haematologic Technologies Inc., Essec Junction, Vt., USA).

### c) Measurement of Anticoagulant Activity

An in vitro assay whereby human blood is collected and added directly to a sodium citrate solution (3.2 g/100 mL, 9 parts blood to 1 part citrate solution). Plasma is prepared by centrifugation (1000 g, 15 minutes) and stored at −80° C.) and an aliquot was rapidly thawed at 37° C. on the day of the experiment and kept on ice before addition to the coagulometer cups. Conventional prothrombin time (PT) tests are carried out in the presence of various concentrations of a test compound and the concentration of test compound required to double the clotting time is determined. Thromborel®S (Dade Behring, Liederbach, Germany) was reconstituted with 10 mL water. This solution was kept at 4° C. and was used within one week. Before the experiment the solution was kept at 37° C. for at least 30 minutes before start of the experiment. A ball coagulation timer KC 10A from Heinrich Amelung GmbH. (Lemgo, Germany) was used to study if the compounds could prevent coagulation in human plasma. The time for 50 μl plasma with compound to coagulate after addition of 100 μl Thromborel S, the Prothrombin Time or PTᵢ, is compared with the time it takes for pure plasma to coagulate, PT₀. With this technique the change in viscosity in the stirred solution is used to define clotting. The IC₅₀ is calculated from the curve of PTᵢ/PTₒ versus the inhibitor concentration in plasma, id est three times the final assay concentration.
d) An in vivo Measurement of Antithrombotic Activity
The abdomen is opened and the caval vein exposed. The thrombotic stimulus is partial stasis to the caval vein and a piece of filter paper soaked with ferric chloride and superimposed to the external surface of the vein. Thrombus size is determined as the thrombus wet weight at the end of the experiment. (Ref Thromb. Res. 2002; 107:163-168).
When tested in the above mentioned screen a) Measurement of Factor Xa Inhibition the compounds of the Examples gave IC₅₀ values for inhibition of Factor Xa activity of less than 10 μM, indicating that the compounds of the invention are expected to possess useful therapeutic properties.
Specimen results are shown in the following Table:

| Compound | IC₅₀ value (nM) |
|---|---|
| Example 4 | 5.0 |
| Example 13 | 2.3 |

A feature of the invention is a compound of formula (I), or a pharmaceutically acceptable salt thereof, for use in medical therapy.

According to a further feature of the invention there is provided a pharmaceutical composition which comprises a compound of formula (I), or a pharmaceutically acceptable salt thereof, in association with a pharmaceutically acceptable diluent or carrier.

The composition may be in a form suitable for oral use, for example a tablet, capsule, aqueous or oily solution, suspension or emulsion; for topical use, for example a cream, ointment, gel or aqueous or oily solution or suspension; for nasal use, for example a snuff, nasal spray or nasal drops; for vaginal or rectal use, for example a suppository; for administration by inhalation, for example as a finely divided powder such as a dry powder, a microcrystalline form or a liquid aerosol; for sub-lingual or buccal use, for example a tablet or capsule; or for parenteral use (including intravenous, subcutaneous, intramuscular, intravascular or infusion), for example a sterile aqueous or oily solution or suspension. In general the above compositions may be prepared in a conventional manner using conventional excipients.

The amount of active ingredient (that is a compound of the formula (I), or a pharmaceutically-acceptable salt thereof) that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient.

According to a further feature of the invention there is provided a compound of formula (I), or a pharmaceutically-acceptable salt thereof, for use in a method of treatment of the human or animal body by therapy.

The invention also includes the use of such an active ingredient (i.e. a compound of the formula (I), or a pharmaceutically-acceptable salt thereof) in the production of a medicament for use in:—
(i) producing a Factor Xa inhibitory effect;
(ii) producing an anticoagulant effect;
(iii) producing an antithrombotic effect;
(iv) treating a Factor Xa mediated disease or medical condition;
(v) treating a thrombosis mediated disease or medical condition;
(vi) treating coagulation disorders; and/or
(vii) treating thrombosis or embolism involving Factor Xa mediated coagulation.

The invention also includes a method of producing an effect as defined hereinbefore or treating a disease or disorder as defined hereinbefore which comprises administering to a warmblooded animal requiring such treatment an effective amount of an active ingredient as defined hereinbefore.

The size of the dose for therapeutic or prophylactic purposes of a compound of the formula (I) will naturally vary according to the nature and severity of the medical condition, the age and sex of the animal or patient being treated and the route of administration, according to well known principles of medicine. As mentioned above, compounds of the formula (I) are useful in the treatment or prevention of a variety of medical disorders where anticoagulant therapy is indicated. In using a compound of the formula (I) for such a purpose, it will generally be administered so that a daily oral dose in the range, for example, 0.5 to 100 mg/kg body weight/day is received, given if required in divided doses. In general lower doses will be administered when a parenteral route is employed, for example a dose for intravenous administration in the range, for example, 0.01 to 10 mg/kg body weight/day will generally be used. For preferred and especially preferred compounds of the invention, in general, lower doses will be employed, for example a daily dose in the range, for example, 0.1 to 10 mg/kg bodyweight/day. In general a preferred dose range for either oral or parenteral administration would be 0.01 to 10 mg/kg body weight/day.

Although the compounds of formula (I) are primarily of value as therapeutic or prophylactic agents for use in warm-blooded animals including man, they are also useful whenever it is required to produce an anticoagulant effect, for example during the ex vivo storage of whole blood or in the development of biological tests for compounds having anticoagulant properties.

The compounds of the invention may be administered as a sole therapy or they may be administered in conjunction with other pharmacologically active agents such as a thrombolytic agent, for example tissue plasminogen activator or derivatives thereof or streptokinase. The compounds of the invention may also be administered with, for example, a known platelet aggregation inhibitor (for example aspirin, a thromboxane antagonist or a thromboxane synthase inhibitor), a known hypolipidaemic agent or a known anti-hypertensive agent.

The compounds of the invention may also be combined and/or co-administered with any antithrombotic agent(s) with a different mechanism of action, such as one or more of the following: the anticoagulants unfractionated heparin, low molecular weight heparin, other heparin derivatives, synthetic heparin derivatives (e.g. fondaparinux), vitamin K antagonists, synthetic or biotechnological inhibitors of other coagulation factors than FXa (e.g. synthetic thrombin, FVIIa, FXIa and FIXa inhibitors, and rNAPc2), the antiplatelet agents acetylsalicylic acid, ticlopidine and clopidogrel; thromboxane receptor and/or synthetase inhibitors; fibrinogen receptor antagonists; prostacyclin mimetics; phosphodiesterase inhibitors; ADP-receptor (P2X1, P2Y1, P2Y12 [P2T]) antagonists; and inhibitors of carboxypeptidase U (CPU or TAFIa) and inhibitors of plasminogen activator inhibitor-1 (PAI-1).
The compounds of the invention may further be combined and/or co-administered with thrombolytics such as one or more of tissue plasminogen activator (natural, recombinant or modified), streptokinase, urokinase, prourokinase, anisoylated plasminogen streptokinase activator complex (APSAC), animal salivary gland plasminogen activators, and the like, in the treatment of tlu-ombotic diseases, in particular myocardial infarction.
The invention further relates to a combination comprising a compound of formula (I) and any antithrombotic agent(s) with a different mechanism of action. Said antitbrombotic agent(s) may be, for example, one or more of the following: the anticoagulants tmfractionated heparin, low molecular weight heparin, other heparin derivatives, synthetic heparin derivatives (e.g. fondaparintix), vitamin K antagonists, synthetic or biotechnological inhibitors of other coagulation factors than FXa (e.g. synthetic thrombin, FVIIa, FXIa and FIXa inhibitors, and rNAPc2), the antiplatelet agents acetylsalicylic acid, ticlopidine and clopidogrel; thromboxane receptor and/or synthetase inhibitors; fibrinogen receptor antagonists; prostacyclin mimetics; phosphodiesterase inhibitors; ADP-receptor (P2X1, P2Y1, P2Y12 [P2T]) antagonists; and inhibitors of carboxypeptidase U (CPU or TAFIa) and inhibitors of plasminogen activator inhibitor-1 (PAI-1).
Moreover, the invention further relates to a combination comprising a compound of formula (I) and thrombolytics, e.g. one or more of tissue plasminogen activator (natural, recombinant or modified), streptokinase, urokinase, prourokinase, anisoylated plasminoger-streptokinase activator complex (APSAC), animal salivary gland plasminogen activators.
Further, the invention also relates to a combination comprising a compound of formula (I) and thrombolytics, e.g. one or more of tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction.

The invention will now be illustrated in the following Examples in which, unless otherwise stated-(i)

Yields are given for illustration only and are not necessarily the maximum attainable. Single node microwave irradiation was performed using either an Emrys Optimizer or a Smith Creator from Personal Chemistry. All solvents and reagents were used as purchased without purification unless noted;

(ii) The end-products have satisfactory high resolution mass spectral (EIRS) data as analysed on a Micromass QT of Micro spectrometer equipped with an Agilent 1100 LC system high performance liquid chromatography (IIPLC). The spectrometer was continually calibrated with leucine enkephaline C₂₈H₃₇N₅O₇ (m/z 556.2771). MS conditions: Electrospray ionization, positive mode, capillary voltage 2.3 kV and desolvation temperature 150° C. Accurate mass was determined for positive ionization using leucine enkephaline (i/z 556.2771) as lock mass. Structures were confirmed by ¹H nuclear magnetic resonance (¹H NMR) spectra which were obtained with either a Varian Unity plus or a Varian Inova spectrometer operating at 400, 500 and 600 MHz respectively. Chemical shift values were measured on the delta scale; the following abbreviations have been used: s, singlet; d, doublet; t, triplet; q, quartet; sept, septet; m, multiplet;

(iii) Isolated intermediates were generally characterised as the end products with the exception of HRMS data;

(iv) Preparative reversed phase HPLC was performed using a Waters Prep LC 2000 with Uw detection equipped with a 25 cm×2 cm or 30×5 cm C8 or C18 columns from Kromasil. Preparative chiral resolution using DPLC was performed using a Gilson 306 with UV detection equipped with either a Ciralpak AS (25×2 cm) (ester separations), a Chiralpak AD (25×2 cm) (amide separations) or a Chirobiotic R (25×2 cm) (carboxylic acid separation) column using 100% methanol or methanol/acetic acid/triethyl amine 100/0.1/0.05. All chiral separations were performed at 40° C.

### EXAMPLE 1

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic Acid

The title product of Example 2, i.e. 4-(3-chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester, (35 mg, 0.061 mmol) was dissolved in tetrahydrofuran (0.75 mL) and a water solution of lithium hydroxide (1 M, 0.25 mL) was added. The mixture was stirred at room temperature for 1 hour. The reaction mixture was neutralized with acetic acid before purification with HPLC using a gradient of acetonitrile/5% acetonitrile water phase containing 0.1 M ammonium acetate, to give 30 mg (88%) of the title compound.

¹H NMR (500 MHz, dimethyl sulfoxide-d₆ as solvent and internal reference) (ppm) 0.88 (dq, 1H, J=4, 12 Hz), 1.02 (dq, 1H, J=4, 12 Hz), 1.23 (broad d, 1H, J=12 Hz), 1.44 (broad d, 1H, J=12 Hz), 1.52-1.62 (m, M1), 2.34-2.54 (m, 3H), 2.98 (dd, 1H, J=4.4, 11.3 Hz), 3.35 (d, 1H, J=16.1 Hz), 3.57-3.70 (m, 5H), 3.77 (dd, 1H, J=3.8, 11.3 Hz), 6.75 (d, 1H, J=10.0 Hz), 7.38 (d, 1H, J=10.0 Hz), 7.46 (dd, 1H, J=1.6, 8.4 Hz), 7.70 (d, 1H, J=8.4 Hz), 7.85-7.87 (m, 2H).

HRMS (ESI+) calc. [M+H]⁺ 563.1474, found 563.1489.

### EXAMPLE 2

### (R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester

### A) (R)-4-(1-Benzenesulfonyl-3-chloro-1H-indole-6-sulfonyl)-1-[1-(1-methyl-6-oxo-1,6-dihydro-pyridazin-3-yl)-piperidin-4-Ylmethyl]-6-oxo-piperazine-2-carboxylic acid methyl ester

To a mixture of (R)-1-[1-(1-methyl-6-oxo-1,6-dihydro-pyridazin-3-yl)-piperidin-4-ylmethyl]-6-oxo-piperazine-2-carboxylic acid methyl ester hydrochloride (185 mg, 0.46 mmol) in anhydrous dichloromethane/N,N-dimethylformamide 5:1 (4 mL) was added pyridine (0.10 mL, 1.2 mmol, 2.5 eq.) at 0° C. under nitrogen atmosphere. To the mixture, a solution of 1-benzenesulfonyl-3-chloro-1H-indole-6-sulfonyl chloride (181 mg, 0.46 mmol, 1.0 eq.) in anhydrous dichloromethane (2 mL) was added at 0° C., and the reaction mixture was stirred at room temperature for 20 minutes. The solvent was removed in vacuo before purification with HPLC using a gradient of acetonitrile/5% acetonitrile-water phase containing 0.1 M ammonium acetate, to give 150 mg (45%) of the sub-title compound after evaporation and freeze drying over night. The sub-title compound was used directly in step B.

### B)

The sub-title compound from step A, i.e. (R)-4-(1-benzenesulfonyl-3-chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester (0.15 g, 0.21 mmol) was dissolved in 3.5 mL anhydrous tetrahydrofuran and a 1 M solution of tetrabutyl-ammonium fluoride (0.23 mmol) in tetrahydrofuran was added. The reaction was heated by single node microwave irradiation at 100° C. for 5 minutes. The solvent was removed in vacuo and the crude was purified by preparative HPLC using a gradient of acetonitrile/5% acetonitrile-water phase containing 0.1 M ammonium acetate. Homogenous fractions were pooled and the main part of acetonitrile was removed in vacuo. Freeze drying in vacuo resulted in the title compound as white solids (62 mg, 51%).

¹H NMR (500 MHz, dimethyl sulfoxide-d₆ as solvent and internal reference) δ (ppm) 0.99 (dq, 1H, J=4, 12 Hz), 1.11 (dq, 1H, J=4, 12 Hz), 1.45 (broad d, 1H, J=12 Hz), 1.56 (broad d, 1H, J=12 Hz), 1.64-1.74 (m, 1H), 2.48-2.64 (m, 3H), 3.01 (dd, 1H, J=3.4, 12.2 Hz), 3.33-3.35 (m, 1H), 3.44 (s, 3H), 3.68 (s, 3H), 3.68-3.77 (m, 3H), 3.81 (d, 1H, J=16.1 Hz), 3.99 (d, 1H, J=12.2 Hz), 4.41 (t, 1H, J=2.7 Hz), 6.75 (d, 1H, J=10.0 Hz), 7.41 (d, 1H, J=10.0 Hz), 7.47 (dd, 1H, J=1.6, 8.4 Hz), 7.72 (d, 1H, J=8.4 Hz), 7.87 (d, 1H, J=1.2 Hz), 7.88 (s, 1H).

HRMS (ESI+) calc. [M+H⁺577.1630, found 577.1622.

### EXAMPLE 3

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid dimethylamide

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, i.e. the title product of Example 1, (50 mg, 0.09 mmol), 2-(7-aza-1H-benzotriazole-1-yl)-1,1,3,3-tetramethyluronium hexafluorophosphate (37 mg, 0.10 mmol) and dimethylamine hydrochloride (22 mg, 0.27 mmol) was dissolved in 2 mL dry N,N-dimethylformamide before N,N-diisopropylethylamine (0.077 mL, 0.44 mmol) was added. The reaction mixture was stirred over night at room temperature. Additional N,N-diisopropyl-ethylamine (1 eq.), dimethylamine hydro chloride (1 eq) and 2-(7-aza-1H-benzotriazole-1-yl)-1,1,3,3-tetramethyluronium hexafluorophosphate (1 eq) was added followed by benzotriazol-1-yl-oxytri-pyrrolidinophosphonium hexafluorophosphate (46 mg, 0.090 mmol). After 2 hours the mixture was purified by preparative HPLC using a gradient of acetonitrile/5% acetonitrile in water buffer containing 0.1 M ammonium acetate to give the product and a byproduct from benzotriazol-1-yl-oxytri-pyrrolidino phosphonium hexafluorophosphate. The crude was dissolved in ethyl acetate and washed three times with 1 M hydrochloric acid and once with water, dried over sodium sulfate, filtered and evaporated in vacuo to give 7.5 mg (14% yield) of the title product as a white powder.

¹H NMR (400 MHz, methanol-d₄ as solvent and internal reference) δ (ppm) 1.18 (m, 2H), 1.52 (broad d, 1H, J=13.0 Hz), 1.65 (broad d, 1H, J=13.0 Hz), 1.73 (m, 1H), 2.49 (m, 1H), 2.63 (m, 2H), 2.85 (s, 3H), 3.06 (s, 3H), 3.16 (m, 1H), 3.49 (d, 1H, J=16.7 Hz), 3.58 (s, 3H), 3.72 (m, 1H), 3.78-3.93 (m, 3H), 4.01 (d, 1H, J=16.7 Hz), 4.66 (m, 1H), 6.79 (d, 1H, J=10.0 Hz), 7.38 (d, 1H, J=10.0 Hz), 7.51 (m, 1H), 7.57 (s, 1H), 7.73 (d, 1H, J=8.3 Hz), 7.9 (s, 1H).

HRMS (ESI+) calc. [M+H]⁺ 590.1953, found 590.1965.

### EXAMPLE 4

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethylamide

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, i.e. the title product of Example 1, (53 mg, 0.09 mmol), triethylamine (0.06 mL, 0.44 mmol) and ethylamine hydrochloride (14 mg, 0.18 mmol) was dissolved in 1.8 mL dry N,N-dimethylformamide. Benzotriazol-1-yl-oxytri-pyrrolidinophosphonium hexafluorophosphate (69 mg, 0.13=mol) was added in one portion. The reaction was stirred for two hours at room temperature. The mixture was purified by preparative HPLC using a gradient of acetonitrile/5% acetonitrile in water buffer containing 0.1 M ammonium acetate to give the product and a by-product from benzotriazole-1-yl-oxy-tris-pyrrolidino-phosphonium hexafluorophosphate. The crude was further purified by flash chromatography on silica gel using dichloromethane/methanol (95:5) as eluent to give the product containing a small amount of byproduct. The crude was dissolved in ethyl acetate and washed with 1 M hydrochloric acid and water, dried over sodium sulfate, filtered and evaporated in vacuo to give pure title product, 25 mg, (45% yield) as a white powder.

¹H NR (400 MHz, methanol-d₄ as solvent and internal reference) δ (ppm) 1.11 (t, 3H, J=7.2 Hz), 1.1-1.3 (m, 2H), 1.49 (broad d, 1H, J=13.3 Hz), 1.61 (broad d, 1H, J=13.3 Hz), 1.75 (m, 1H), 2.49-2.66 (m, 3H), 3.13 (m, 1H), 3.20 (q, 2H, J=7.2 Hz), 3.46 (d, 1H, J=16.1 Hz), 3.57 (s, 3H), 3.76-3.93 (m, 4H), 4.0 (d, 1H, J=16.1 Hz), 4.09 (m, 1H), 6.79 (d, 1H, J=9.3 Hz), 7.38 (d, 1H, J=9.3 Hz), 7.51 (m, 1H), 7.57 (s, 1H), 7.73 (d, 1H, J=8.6 Hz), 7.90 (s, 1H).

HRMS (ESI+) calc. [M+H]⁺ 590.1953, found 590.1959.

### EXAMPLE 5

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-hydroxy-ethyl)-amide

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, i.e. the title product of Example 1, (50 mg, 0.090 mmol), triethylamine (0.10 mL, 0.72 mmol) and ethanol amine (11 mg, 0.18 mmol) was dissolved in 1.8 mL dry N,N-dimethylformamide. Benzotriazol-1-yl-oxytri-pyrrolidinophosphonium hexafluorophosphate (69 mg, 0.13 mmol) was added in one portion. The reaction was stirred over night at room temperature. The mixture was purified by preparative HPLC using a gradient of acetonitrile/5% acetonitrile in water buffer containing 0.1 M ammonium acetate to give 42 mg (78% yield) of the desired title product after freeze drying over night.

¹H NMR (300 MHz, acetic acid-d₄ as solvent and internal reference) δ (ppm) 1.24 (m, 2H), 1.48-1.68 (m, 2H), 1.89 (m, 1H), 2.67 (m, 3H), 3.12 (m, 1H), 3.49 (t, 2H, J=5.2 Hz), 3.58 (d, 1H, J=16.7 Hz), 3.66 (s, 3H), 3.79 (t, 2H, J=5.2 Hz), 3.84-4.0 (m, 3H), 4.10 (m, 1H), 4.19 (d, 1H, J=16.7 Hz), 4.36 (m, 1H), 7.10 (d, 1H, J=9.4 Hz), 7.34 (d, 1H, J=9.4 Hz), 7.55 (m, 2H), 7.75 (d, 1H, J=7.7 Hz), 7.99 (m, 1H).

HRMS (ESI+) calc. [M+H]⁺ 606.1901, found 606.193.

### EXAMPLE 6

### 6-{4-[4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one

### i) 6-{4-[(R)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one and ii)-6-{4-[(S)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, i.e. the title product of Example 1, (78 mg, 0.14 mmol) and morpholine (0.050 mL, 0.57 mmol) was dissolved in 1.5 mL dry N,N-dimethylformamide, 2-(1H-benzotriazole-1-yl)-1,1,3,3-tetramethyl-uronium tetrafluoroborate (54 mg, 0.17 mmol) was added in one portion. The reaction was stirred for 4 hours at room temperature. More 2-(1H-benzotriazole-1-yl)-1,1,3,3-tetramethyluronium tetrafluoroborate (25 mg, 0.080 mmol) was added and the mixture was stirred for 1 hour. The crude mixture was purified by preparative HPLC using acetonitrile/5% acetonitrile in water buffer containing 0.1 M ammonium acetate to give 60 mg (68% yield) of the title compound as a light yellow powder after evaporation of solvent and freeze drying over night.

¹H NMR (400 MHz, methano-d₄ as solvent and internal reference) (ppm) 1.19 (m, 2H), 1.54 (broad d, 1H, J=12.9 Hz), 1.66 (broad d, 1H, J=12.9 Hz), 1.75 (m, 1H), 2.51 (m, 1H), 2.63 (m, 2H), 3.07 (m, 1H), 3.42 (m, 2H), 3.49-3.94 (m, 14H), 4.04 (d, 1H, J=16.7 Hz), 4.64 (m, 1H), 6.79 (d, 1H, J=9.8), 7.38 (d, 1H, J=9.8 Hz), 7.51 (m, 1H), 7.57 (s, 1H), 7.73 (d, 1H, J=8.2 Hz), 7.90 (s, 1H).

The enantiomers i) and ii) were separated by preparative chiral chromatography.

i) HRMS (ESI+) calc. [M+H]⁺ 632.2058, found 632.2092.
ii) HRMS (ESI+) calc. [M+H]⁺ 632.2058, found 632.2092.

### EXAMPLE 7

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide

i) (R-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide and ii) (S)-4-(3-Chloro-1H-indole-6-sulfonyl)-1-[1-(1-methyl-6-oxo-1,6-dihydro pyridazin-3-yl)-piperidin-4-ylmethyl]-6-oxo-piperazine-2-carboxylic acid isopropylamide

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, the title product of Example 1 (54 mg, 0.096 mmol) was dissolved in 1 ml dry N,N-dimethylformamide, diisopropyl-ethylamine (0.031 mL, 0.18 mmol) and 2-(1H-benzotriazole-1-yl)-1,1,3,3-tetramethyl-uronium tetrafluoroborate (34 mg, 0.11 mmol) was added. The mixture was stirred for 5 minutes at room temperature before N,N-diisopropylamine (0.030 mL, 0.35 mmol) was added. The reaction mixture was stirred over night. More N,N-diisopropylethylamine (0.10 mL, 0.57 mmol), 2-(1H-benzotriazole-1-yl)-1,1,3,3-tetramethyluronium tetra-fluoroborate (31 mg, 0.096 mmol) and isopropylamine (0.10 mL, 1.2 mmol) was added. After 2 days part of the solvent was evaporated in vacuo and the crude was purified by preparative HPLC using a gradient of acetonitrile/5% acetonitrile in water phase containing 0.1 M ammonium acetate to give 31 mg (53% yield) of the desired title compound as a white powder after evaporation of solvent and freeze drying over night.

¹H NMR (400 MHz, methanol-d₄ as solvent and internal reference) δ (ppm) 1.11-1.24 (m, 8H), 1.49 (broad d, 1H, J=12.8 Hz), 1.62 (broad d, 1H, J=12.8 Hz), 1.74 (m, 1H), 2.59 (m, 3H), 3.15 (m, 1H), 3.46 (d, 1H, J=16.1 Hz), 3.58 (s, 3H), 3.73-3.87 (m, 4H), 3.93-4.01 (m, 2H), 4.08 (m, 1H), 6.79 (d, 1H, J=9.8 Hz), 7.38 (d, 1H, J=9.8 Hz), 7.51 (m, 1H), 7.57 (s, 1H), 7.73 (d, 1H, J=8.5 Hz), 7.90 (s, 1H).

The enantiomers i) and ii) were separated by preparative chiral chromatography.

i) HRMS (ESI+) calc. [M+H]⁺ 590.1953, found 590.1964.

### EXAMPLE 8

### 6-{4-[2-(Azetidine-1-carbonyl)-4-(3-chloro-H-indole-6-sulfonyl)-6-oxo-piperazin-1-ylmethyl]-piperidin-1-yl}-2-methyl-2H-pyridazin-3-one

### i) 6-{4-[(R)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one and ii) 6-{4-[(S)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, the title product of Example 1 (62 mg, 0.11 mmol), was dissolved in 1.1 mL N,N-dimethylformamide, N,N-diisopropylethylamine (0.038 mL, 0.22 mmol) and 2-(1H-benzotriazole-1-yl)-1,1,3,3-tetramethyluronium tetrafluoroborate (39 mg, 0.12 mmol) was added. The mixture was stirred for 5 minutes before azetidine (0.03 mL, 0.44 mmol) was added. The reaction mixture was stirred over night. More N,N-diisopropylethylamine (0.1 mL, 0.57 mmol), 2-(1H-benzotriazole-1-yl)-1,1,3,3-tetramethyluronium tetrafluoroborate (40 mg, 0.12 mmol) and azetidine (0.03 mL, 0.44 mmol) was added. After 2 days, part of the solvent was evaporated in vacuo and the crude was purified by preparative HPLC using a gradient of acetonitrile/5% acetonitrile in water phase containing 0.1 M ammonium acetate to give 39 mg (58% yield) of the desired title compound as a light yellow powder after evaporation of solvent and freeze drying over night.

¹H NMR (400 MHz, methanol-d₄ as solvent and internal reference) δ (ppm) 1.17 (i, 2H), 1.50 (broad d, 1H, J=12.2 Hz), 1.62 (broad d, 1H, J=12.2 Hz), 1.73 (m, 1H), 2.25 (m, 2H), 2.59 (m, 3H), 3.18 (m, 1H), 3.54 (d, 1H, J=16.4 Hz), 3.58 (s, 3H), 3.72-3.86 (m, 5H), 4.00 (m, 2H), 4.14 (m, 1H), 4.21-4.31 (m, 2H), 6.80 (d, 1H, J=10.1 Hz), 7.38 (d, 1H, J=10.1 Hz), 7.55 (m, 1H), 7.58 (s, 1H), 7.75 (d, 1H, 8.8 Hz), 7.93 (s, 1H).

The enantiomers i) and ii) were isolated by preparative chiral chromatography.

i) HRMS (ESI+) calc. [M+H]⁺ 602.1953, found 602.1948.
ii) HRMS (ESI+) calc. [M+H]⁺ 602.1953, found 602.1958.

### EXAMPLE 9

### 6-{4-[4-(3-Chloro-1H-indole-6-sulfonyl)-2-hydroxymethyl-6-oxo-piperazin-1-ylmethyl]-piperidine-1-yl}-2-methyl-2H-pyridazin-3-one

### A) 6-{4-[4-(1-Benzenesulfonyl-3-chloro-1H-indole-6-sulfonyl)-2-hydroxymethyl-6-oxo-piperazin-1-ylmethyl]-piperidin-1-yl}-2-methyl-2H-pyridazin-3-one

4-(1-Benzenesulfonyl-3-chloro-1H-indole-6-sulfonyl)-1[1-(1-methyl-6-oxo-1,6-dihydro-pyridazine-3-yl)-piperidin-4-ylmethyl]-6-oxo-piperazine-2 carboxylic acid, the title product of Example 1 (30 mg, 0.040 mmol) was dissolved in tetrahydrofuran (5 ml) together with triethylamine (5 mg, 0.05 mmol). The reaction mixture was cooled on an ice/salt bath to −18° C. and isobutyl chloroformate (6 mg, 0.05 mmol) was added. After 30 minutes the formed precipitate was filtered off and the reaction mixture was cooled again to −18° C. Sodium borohydride (5 mg, 0.13 mmol) was added and a few drops of water. When the foaming was over another 2 mL of water was added and the reaction mixture was allowed to stand at ambient temperature for 1 hour. Water was added, tetrahydrofuran was removed in vacuo and the remaining water phase was extracted three times with dichloromethane. The combined organic phase was washed with water and brine, dried with sodium sulfate and after filtration the solvent was evaporated in vacuo to give 30 mg of the sub-title compound which was used without further purification in the next step.

### B)

The intermediate was dissolved in tetrahydrofuran (2 mL) and lithium hydroxide (2 mg, 0.09 mmol) dissolved in water (1 mL) was added. The reaction mixture was allowed to stand at ambient temperature for 2 hours whereupon the pH was adjusted to 5-6 by addition of 0.1 M hydrochloric acid. Water (20 mL) was added, tetrahydrofuran was removed in vacuo and the remaining water phase was extracted three times with dichloromethane (20 mL). The combined organic phase was washed with water and brine, dried with sodium sulfate and the solvent evaporated in vacuo. The residue was purified by HPLC (Kromasil C8) using a gradient of acetonitrile (20-70% in water containing 0.1 M ammonium acetate to give 4.5 mg of the title compound after evaporation and freeze drying.

¹H NMR (400 MHz, methanol-d₄ as solvent and internal reference) δ (ppm): 1.04-1.20 (m, 1H), 1.19-1.30 (m, 1H), 1.45-1.52 (broad d, 1H), 1.58-1.65 (broad d, 1H), 1.81-1.90 (m, 1H), 2.62 (q, 2H, J=12 Hz), 2.80-2.91 (m, 2H), 3.38 (d, 1H, J=17.6 Hz), 3.46-3.52 (m, 1H), 3.59 (s, 3H), 3.67-3.76 (m, 2H), 3.77-3.89 (m, 3H), 3.90-3.98 (m, 2H), 6.81 (d, 1H, J=10 Hz), 7.38 (d, 1H, J=10 Hz), 7.55 (d, 1H, J=8.8 Hz), 7.58 (s, 1H), 7.76 (d, 1H, J=8.8 Hz), 7.94 (s, 1H).

HRMS (ESI+) calc. [M+H]+ 549.1687, found 549.1686.

### EXAMPLE 10

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide

### i) (R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-ethoxy-ethyl)-amide and ii) (S)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid, the title product of Example 1, (40 mg, 0.071 mmol) was dissolved in 1 mL dry N,N-dimethylformamide and 2-(1H-benzotriazole-1-yl)-1,1,3,3-tetramethyluronium tetrafluoroborate (91 mg, 0.28 mmol, 4 equiv.) was added. The mixture was stirred for 5 minutes at room temperature before 2-methoxy-ethylamine (0.031 ml, 0.36 mmol) was added. The reaction mixture was stirred for 1 hour. The crude material was purified by preparative HPLC using a gradient of acetonitrile/5% acetonitrile in water phase containing 0.1 M ammonium acetate to give 40 mg (91% yield) of the desired title compound as a white powder after evaporation of solvent and freeze drying over night.

¹H NMR (500 MHz, methanol-d₄ as solvent and internal reference) (ppm) 1.10-1.27 (m, 2H), 1.50 (broad d, 1H, J=13 Hz), 1.63 (broad d, 1H, J=13 Hz), 1.72-1.82 (, 1H), 2.55-2.69 (m, 3H), 3.14-3.20 (m, 1H), 3.35 (s, 3H), 3.36-3.52 (m, 5H), 3.59 (s, 3H), 3.77-3.92 (m, 4H), 4.01 (d, 1H, J=17 Hz), 4.14-4.18 (m, 1H), 6.81 (d, 1H, J=10 Hz), 7.40 (d, 1H, J=10 Hz), 7.51-7.55 (m, 1H), 7.59 (s, 1H), 7.75 (d, 1H, J=9 Hz), 7.92 (s, 1H).

The enantiomers i) and ii) were isolated by preparative chiral chromatography.

i) FIRMS (ESI+) calc. [M+H]⁺620.2058, found 602.2055.
ii) HRMS (ESI+) calc. [M+H]⁺ 620.2058, found 602.2056.

### EXAMPLE 11

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid tert-butyl ester

4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (50 mg, 0.089 mmol) was suspended in dry toluene (1.5 ml). N,N-dimethylformamide di-tert-butyl acetal (72 mg, 0.36 mmol, 4 equiv.) was added dropwise before the reaction mixture was heated at 85° C. (oil bath temperature). One equivalent of N,N-dimethylformamide di-tert-butyl acetal was added dropwise. The reaction mixture was stirred for an additional hour. This procedure was repeated twice. The reaction mixture was cooled and concentrated under reduced pressure before purification by prep-HPLC using a gradient of acetonitrile/5% acetonitrile in a water phase containing 0.1 M ammonium acetate to give 15 mg (27% yield) of the desired title compound as a white powder after evaporation of solvent and freeze drying over night.

¹H NMR (500 MHz, dimethyl sulfoxide-d₆ as solvent and internal reference) δ (ppm) 0.96-1.17 (m, 2H), 1.43-1.48 (m, 10H), 1.57 (broad d, 1H, J=14 Hz), 1.62-1.72 (m, 1H), 2.46-2.58 (m, 3H), 2.90 (dd, 1H, J=3, 12 Hz), 3.22 (d, 1H, J=16 Hz), 3.44 (s, 3H), 3.63-3.82 (m, 4H), 4.00 (d, 1H, J=12 Hz), 4.24-4.27 (m, 1H), 6.75 (d, 1H, J=10 Hz), 7.41 (d, 1H, J=10 Hz), 7.48 (dd, 1H, J=2, 8 Hz), 7.72 (d, 1H, J=8 Hz), 7.87 (d, 1H, J=1 Hz), 7.88 (s, 1H).

HRMS (ESI+) calc. [M+H]⁺ 619.2106, found 619.207.

### EXAMPLE 12

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethyl ester

To a reaction vial containing 4-(3-chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (12 mg, 0.021 mmol) was added hydrochloric acid-saturated ethanol. The reaction vial was equipped with a septum and the reaction mixture was heated at 70° C. for 90 min. The reaction mixture was evaporated to dryness under reduced pressure before the crude was dissolved in dimethyl sulfoxide and purified by preparative HPLC using a gradient of acetonitrile/5% acetonitrile in a water phase containing 0.1 M ammonium acetate to give 12 mg (95% yield) of the desired title compound as a white powder after evaporation of solvent and freeze drying over night.

¹HNMR (500 MHz, acetonitrile-d₃ as solvent and internal reference) δ (ppm) 1.08 (dq, 1H, J=4, 12 Hz), 1.18 (dq, 1H J=4, 12 Hz), 1.25 (t, 3H, J=7 Hz), 1.49 (broad d, 1H, J=13 Hz), 1.59 (broad d, 1H, J=13 Hz), 1.63-1.73 (m, 1H), 2.50-2.59 (m, 3H), 2.94 (dd, 1H, J=3, 12 Hz), 3.32 (d, 1H, J=16 Hz), 3.48 (s, 3H), 3.65-3.76 (m, 2H), 3.81 (dd, 1H, J=8, 14 Hz), 3.93 (d, 1H, J=16 Hz), 4.10 (dim, 1H, J=12 Hz), 4.12-4.24 (m, 3H), 6.67 (d, 1H, J=10 Hz), 7.18 (d, 1H, J=10 Hz), 7.52 (dd, 1H, J=1, 8 Hz), 7.57 (d, 1H, J=3 Hz), 7.74 (d, 1H, J=8 Hz), 7.95 (s, 1H), 9.96 (s, 1 NH).

HRMS (ESI+) calc. [M+H]⁺ 591.1793, found 591.1782.

### EXAMPLE 13

### 4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropyl ester

To a reaction vial containing 4-(3-chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (180 mg, 0.32 mmol) was added hydrochloric acid-saturated propan-2-ol. The reaction vial was equipped with a septum and the reaction mixture was heated at 85° C. for 2.5 h. The reaction mixture was evaporated to dryness under reduced pressure before the crude was dissolved in dimethyl sulfoxide and purified by preparative HPLC using a gradient of acetonitrile/5% acetonitrile in a water phase containing 0.1 M ammonium acetate to give 144 mg (74% yield) of the desired title compound as a white powder after evaporation of solvent and freeze drying over night.

¹H NMR (500 MHz, dimethyl sulfoxide-d₆ as solvent and internal reference) δ (ppm) 1.00 (dq, 1H, J=4, 12 Hz), 1.12 (dq, 1H J=4, 12 Hz), 1.24 (dd, 6H, J=2, 6 Hz), 1.46 (broad d, 1H, J=12 Hz), 1.57 (broad d, 1H, J=12 Hz), 1.64-1.74 (m, 1H), 2.48-2.59 (m, 3H), 2.95 (dd, 1H, J=3, 12 Hz), 3.25 (d, 1H, J=16 Hz), 3.44 (s, 3 H), 3.65-3.77 (m, 3H), 3.79 (dd, 1H, J=16 Hz), 4.00 (d, 1H, J=12 Hz), 4.33-4.36 (m, 1H), 4.98 (sept., 1H, J=6 Hz), 6.75 (d, 1H, J=10 Hz), 7.41 (d, 1H, J=10 Hz), 7.47 (dd, 1H, J=2, 8 Hz), 7.72 (d, 1H, J=8 Hz), 7.87 (d, 1H, J=1 Hz), 7.88 (s, 1H).

HRMS (ESI+) calc. [M+H]⁺ 605.1949, found 605.1946.

1. A compound of formula (I) 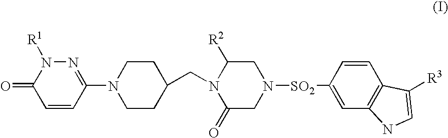
wherein
R¹ is hydrogen or C₁₋₃alkyl;
R² is selected from hydroxy, C₁₋₅alkyl, carboxy, cyano, tetrazolyl, N—C₁₋₅ alkyltetrazolyl, oxazolyl, C₁₋₅ oxazolyl, isoxazolyl, C₁₋₅ isoxazolyl, hydroxyC₁₋₅alkyl, carboxyC₁₋₅alkyl, C₁₋₅alkoxyoxoC₁₋₅alkyl, carbamoyl, C₁₋₅alkylcarbamoyl, di(C₁₋₅alkyl)carbamoyl, C₁₋₁₅alkylcarbamoylC₁₋₄alkyl, hydroxyC₁₋₅alkylcarbamoyl, C₁₋₅alkoxyC₁₋₅alkylcarbamoyl; —C₁₋₅alkyl-Y¹, —COOCHR¹⁷R¹⁸ and —CONR¹⁷R¹⁸:
wherein
Y¹ represents O(CH₂)ᵣR¹⁴,
r represents an integer 1 to 4;
when r represents an integer 2 to 4, R¹⁴ represents hydroxy, C₁₋₅alkylalkoxy, carboxy, C₁₋₅alkoxycarbonyl, S(O)ₚR⁹ or NR¹⁵R¹⁶; and when r represents 1, R¹⁴ represents carboxy or
C₁₋₅alkoxycarbonyl;
wherein any phenyl group within R² is independently substituted by 0, 1 or 2 substituents selected from halogeno, trifluoromethyl, cyano, C₁₋₅alkyl and C₁₋₅alkoxy;
p is 0, 1 or 2;
R⁹ represents C₁₋₅alkyl or phenyl;
R¹⁵ and R¹⁶ independently represent hydrogen or C₁₋₅alkyl;
R¹⁷ and R¹⁸ are independently selected from hydrogen, C₁₋₆alkyl, C₄₋₇cycloalkyl, C₂₋₆alkenyl, R¹⁷ and R¹⁸ may form, along with the carbon to which they are attached, a 4-, 5-, 6- or 7-membered carbocyclic ring which contains 0, 1 or 2 heteroatoms selected from nitrogen, oxygen and sulphur, or R¹⁷ and R¹⁸ may form, along with the nitrogen to which they are attached, a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl; and
R³ is hydrogen or halogen;
or a pharmaceutically acceptable salt thereof.

2. A compound according to claim 1 where R¹ is C₁₋₃alkyl.

3. A compound according to claim 1 where R¹ is methyl.

4. A compound according to claim 1, wherein R² is selected from hydroxy, C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —C₁₋₅alkyl-Y¹, —COOCHR¹⁷R¹⁸ and —CONR¹⁷R¹⁸:
wherein
Y¹ represents O(CH₂)ᵣR¹⁴;
r represents an integer 1 to 4;
when r represents an integer 2 to 4, R¹⁴ represents hydroxy, C₁₋₅alkylalkoxy, carboxy, C₁₋₅alkoxycarbonyl, S(O)ₚR⁹ or NR¹⁵R¹⁶; and when r represents 1, R¹⁴ represents carboxy or C₁₋₅alkoxycarbonyl;
wherein any phenyl group within R² is independently substituted by 0, 1 or 2 substituents selected from halogeno, trifluoromethyl, cyano, C₁₋₅alkyl and C₁₋₅alkoxy;
p is 0, 1 or 2;
R⁹ represents C₁₋₅alkyl or phenyl;
R¹⁵ and R¹⁶ independently represent hydrogen or C₁₋₅alkyl;
R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

5. A compound according to claim 1 where R² is selected from hydroxy, C₁₋₃alkyl, carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl, C₁₋₅alkoxyC₁₋₅alkylcarbamoyl, —COOCHR¹⁷R¹⁸ and —CONR¹⁷R¹⁸:
wherein
R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0, 1 or 2 additional heteroatoms selected from nitrogen, oxygen and sulphur, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

6. A compound according to claim 1, wherein R² is selected from carboxy, hydroxyC₁₋₅alkyl, C₁₋₅alkoxyoxoC₁alkyl, carbamoyl, C₁₋₅alkylcarbamoyl, di(C₁₋₅alkyl)carbamoyl, hydroxyC₁₋₅alkylcarbamoyl and C₁₋₅alkoxyC₁₋₅alkylcarbamoyl.

7. A compound according to claim 1, wherein R² is selected from —COOCHR¹⁷R¹⁸ and —CONR¹⁷R¹⁸: R¹⁷ and R¹⁸ are independently selected from hydrogen, C₁₋₆alkyl, C₄₋₇cycloalkyl, C₂₋₆alkenyl, R¹⁷ and R¹⁸ may form along with the carbon to which they are attached a 4-, 5-, 6- or 7-membered carbocyclic ring which contains 0, 1 or 2 heteroatoms selected from nitrogen, oxygen and sulphur, or R¹⁷ and R¹⁸ may form along with the nitrogen to which they are attached a 4-, 5-, 6- or 7-membered heterocyclic ring which contain in addition to the nitrogen atom present 0 or 1 additional hetero oxygen, wherein each R¹⁷, R¹⁸ or any of said rings formed by R¹⁷ and R¹⁸ is independently substituted by 0, 1 or 2 substituents selected from hydroxy, amino, carboxy, C₁₋₅alkoxycarbonyl, oxo, C₁₋₅alkyl, hydroxyC₁₋₅alkyl, C₁₋₅alkoxyC₁₋₅alkyl, carboxyC₁₋₅alkyl, C₁₋₅alkoxyoxoC₁₋₆alkyl, and carbamoylC₁₋₅alkyl.

8. A compound according to claim 1 wherein R³ is halogen.

9. A compound according to claim 1 which is
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid methyl ester,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid dimethylamide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethylamide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-hydroxy-ethyl)-amide,
6-{4-[4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
6-{4-[(R)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
6-{4-[(S)-4-(3-Chloro-1H-indole-6-sulfonyl)-2-(morpholine-4-carbonyl)-6-oxo-piperazin-1-ylmethyl]-piperidin-1-yl}-2-methyl-2H-pyridazin-3-one,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropylamide,
(S)-4-(3-Chloro-1H-indole-6-sulfonyl)-1-[1-(1-methyl-6-oxo-1,6-dihydro pyridazin-3-yl)-piperidin-4-ylmethyl]-6-oxo-piperazine-2-carboxylic acid isopropylamide,
6-{4-[2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[(R)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[(S)-2-(Azetidine-1-carbonyl)-4-(3-chloro-1H-indole-6-sulfonyl)-6-oxo-piperazin-1-ylmethyl]-piperidin-1-yl}-2-methyl-2H-pyridazin-3-one,
6-{4-[4-(3-Chloro-1H-indole-6-sulfonyl)-2-hydroxymethyl-6-oxo-piperazin-1-ylmethyl]-piperidine-1-yl}-2-methyl-2H-pyridazin-3-one,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
(R)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
(S)-4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid (2-methoxy-ethyl)-amide,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid tert-butyl ester,
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid ethyl ester, or
4-(3-Chloro-1H-indole-6-sulfonyl)-1-[1-(1-methyl-6-oxo-1,6-dihydro-pyridazin-3-yl)-piperidin-4-ylmethyl]-6-oxo-piperazine-2-carboxylic acid isopropyl ester.

10. A process for preparing a compound of formula (I) as defined in claim 1 which process comprises either
(a) where an amide derivative from the exocyclic carboxylic acid of formula (II), 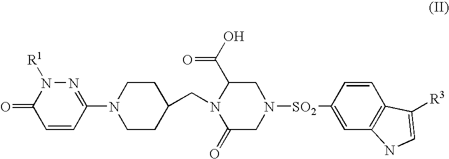
or a reactive derivative thereof, are prepared, using an primary or secondary amine or a salt thereof, wherein R¹ and R³ are as defined in claim 1, and reacting with and an inorganic acid chloride;
(b) where the compound of formula (I) is an ester derivative of the compound of formula (II), the compound of formula (II) is treated in a readily available alcoholic solvent using acid catalysis, and using in the case of hindered alcohols N,N-dimethylformamide dialkyl acetal;
(c) reacting a sulfonyl chloride derivative of formula (III), with or without a protecting group on the indolyl nitrogen, 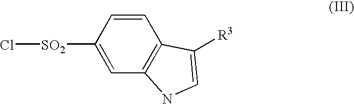
wherein R³ is as defined in claim 1 with an amine of formula (IV), 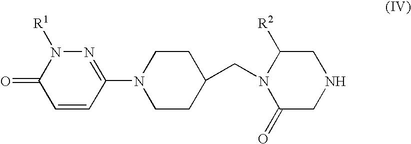
or a salt thereof, wherein R¹ and R² are as defined in claim 1; or
(d) reacting a carboxylic acid derivative of formula (IV), or a reactive intermediate thereof, followed by addition of a reducing agent.

11. (canceled)

12. A pharmaceutical composition comprising a compound of formula (I), or a pharmaceutically-acceptable salt thereof, as defined in claim 1, with a pharmaceutically-acceptable diluent or carrier.

13. (canceled)

14. A method of treating a Factor Xa mediated disease or condition in a warm-blooded animal comprising administering an effective amount of a compound of formula (I), as defined in claim 1, or a pharmaceutically-acceptable salt thereof.

15. A combination comprising a compound of formula (I), as defined in claim 1, or a pharmaceutically-acceptable salt thereof, and one or more antithrombotic agent(s) with a different mechanism of action, wherein said antithrombotic agent(s) is selected from: an anticoagulant, a vitamin K antagonist, a synthetic or biotechnological inhibitor of other coagulation factors than FXa an antiplatelet agent; a thromboxane receptor and/or synthetase inhibitor; a fibrinogen receptor antagonist; a prostacyclin mimetic; a phosphodiesterase inhibitor; an ADP-receptor antagonist; and an inhibitor of carboxypeptidase U and an inhibitor of plasminogen activator inhibitor-1 (PAI-1).

16. A combination comprising a compound of formula (I), as defined in claim 1, or a pharmaceutically-acceptable salt thereof, and a thrombolytic agent.

17. A process according to claim 10 wherein:
the alcoholic solvent using acid catalysis in (b) is saturation of the solvent by gaseous hydrogen chloride; and
the reactive intermediate in (d) is a mixed anhydride formed by reacting (IV) with an alkyl chloroformate in situ.

18. A combination according to claim 15 wherein:
the anticoagulant is unfractionated heparin, low molecular weight heparin, other heparin derivative, or a synthetic heparin derivative;
a synthetic or biotechnological inhibitor of other coagulation factors than FXa is an inhibitor of synthetic thrombin, FVIIa, FXIa, FIXa, or rNAPc2;
the antiplatelet agent is acetylsalicylic acid, ticlopidine, or clopidogrel;
the ADP-receptor antagonist is an antagonist of P2X1, P2Y1, P2Y12, or P2T; and
the carboxypeptidase U is CPU or TAFIa.

19. A combination according to claim 18 wherein the synthetic heparin derivative is fondaparinux.

20. A combination according to claim 16 wherein the thrombolytic agent is selected from a tissue plasminogen activator, streptokinase, urokinase, prourokinase, anisoylated plasminogen-streptokinase activator complex (APSAC), or an animal salivary gland plasminogen activator.
